(19) United States
(12) Patent Application Publication
(10) Pub. No.: US 2011/0306865 A1
(43) Pub. Date: Dec. 15, 2011

(54) PHOTOACOUSTIC IMAGING DEVICE

(75) Inventors: Michael M. Thornton, London (CA); Robert A. Kruger, Oriental, NC (US)

(73) Assignee: ENDRA, INC.

(21) Appl. No.: 13/063,259

(22) PCT Filed: Sep. 10, 2009
(86) PCT No.: PCT/US09/56563

§ 371 Date: Aug. 5, 2011

Related U.S. Application Data
(60) Provisional application No. 61/095,881, filed on Sep. 10, 2008.

(51) Int. Cl. A61B 6/00

(52) U.S. Cl. 600/407

(57) ABSTRACT

The invention features a system for imaging tissue including (i) a source of electromagnetic radiation; (ii) an encasement h a plurality of acoustic transducers (e.g., at least 128); (iii) a support structure having a portion for holding a tissue; and (iv) a chamber between the encasement and support structure for housing an acoustic coupling medium. In the system, electromagnetic radiation from the source is sufficient to induce a thermoacoustic response in the tissue positioned in the support structure, and the plurality of acoustic transducers are positioned to receive ultrasound from the thermoacoustic response of the tissue. The invention also features methods of imaging a tissue using the systems.

### CROSS-REFERENCE TO RELATED APPLICATIONS

This application claims benefit of U.S. Provisional Application No. 61/095,881, filed Sep. 10, 2008, which is hereby incorporated by reference.

### FIELD OF THE INVENTION

The present invention relates generally to a system and method for acoustic imaging and more particularly to a diagnostic photo-acoustic imaging system and method for low volume imaging.

### BACKGROUND OF THE INVENTION

Non-Invasive Small Animal Imaging

Low volume imaging relates to diagnostic imaging tailored to low volume objects. Low volume imaging has applications in human diagnostic imaging of smaller body parts, including wrist, hand, and foot. It has further application in tissue specimen imaging and preclinical (i.e., non-human animal) imaging.

Preclinical models of disease have become more available and sophisticated. They are now a common tool used in the development and evaluation of new therapeutics and treatments. The use of preclinical models is a precursor and validation step prior to human clinical trials.

Non-invasive imaging is an important tool in preclinical studies; computed tomography (CT), magnetic resonance imaging (MR), single photon emission tomography (SPECT), positron emission tomography (PET), x-ray, optical, and ultrasound are standard tools in studying disease and the evaluation of new therapies. These imaging tools are being actively used for understanding and assisting in therapy development of diseases, such as cardiovascular, musculoskeletal, neoplasia, auto immune, and inflammation.

Clinical imaging devices are often sufficient for imaging of large animal species such as primates, porcine, and canine. However, the vast majority of preclinical studies involve the use of lower volume animals, such as rodents and rabbits; where the murine model (mouse) is the most widely used preclinical model.

In recent years specialized devices have been developed and commercialized specifically for low volume animal imaging based on standard non-invasive medical imaging technologies.

The aim of low volume imaging is to have the full functionality of clinical imaging, but with sensitivity and resolution at the scale of the low volume object of interest. Clinical imaging systems are not directly scalable to low volume imaging systems. As such, in all of the above mentioned imaging modalities, technical and scientific hurdles had to be overcome to achieve systems that had proper functioning, often including achieving higher resolution, the need for miniaturization of many aspects of the technology, and proper placement and handling of low volume objects, specifically animals.

As in clinical imaging, each of the preclinical imaging modalities helps to visualize different aspects of an object and has different strengths. Some of the desirable attributes include sensitivity, resolution, field of view, minimal required time to produce an image, contrast, cost, three-dimensional imaging, and whether the system is capable of dynamic imaging. No one imaging modality is sufficient for all applications. Furthermore, the current array of low volume imaging modalities still do not provide the full functionality in visualization and quantification that is desired for current preclinical and other low volume needs.

Photo/Thermo-Acoustic Imaging

Two relatively new imaging technologies are thermo-acoustic and photo-acoustic imaging (collectively referred to herein as photo-acoustic imaging). This new modality adds new insights into properties of tissues and other objects, above those offered by established imaging modalities. Specifically, it provides information related to the thermoelastic properties of tissue. More specifically, laser, radio frequency or other energy pulses are delivered into an object. Some of the delivered energy will be absorbed and converted into heat, leading to transient thermoelastic expansion and thus ultrasonic emission. The generated ultrasonic waves are then detected by ultrasonic transducers to form images (Bowen, Radiation-Induced Thermoacoustic Soft Tissue Imaging, Proc. of IEEE Ultrasonic Symposium 2:817-822, June, 1981).

No system currently exists that is ideally suited for low volume photo-acoustic imaging. Accordingly, there is a need for new low-volume photoacoustic imaging systems.

### SUMMARY OF THE INVENTION

In general, the invention features systems for imaging tissue and methods of their use.

In one aspect, the invention features a system for imaging tissue including (i) a source of electromagnetic radiation; (ii) an encasement h a plurality of acoustic transducers (e.g., at least 128); (iii) a support structure having a portion for holding a tissue; and (iv) a chamber between the encasement and support structure for housing an acoustic coupling medium. In the system, electromagnetic radiation from the source is sufficient to induce a thermoacoustic response in the tissue positioned in the support structure, and the plurality of acoustic transducers are positioned to receive ultrasound from the thermoacoustic response of the tissue. In addition, the portion for holding the tissue has a thickness of less than 250 μm, and the acoustic impedance of the portion is matched to the tissue (i.e., within 50-150% of that of the tissue), or the portion allows for contact between the tissue and the acoustic coupling medium.

The system may further include one or more of an optical camera (e.g., sensitive to light from 300-1064 nm) positioned to monitor the tissue in the support structure; an electro-mechanical motion control system for rotation of the encasement relative to the support structure (e.g., in movements of 1 degree or less); a digital acquisition system for acquiring and storing thermoacoustic response signals received by the plurality of transducers; a temperature monitor and control system for maintaining a specified temperature (e.g., between 30 and 39° C.) of acoustic coupling medium in the chamber; and a pulse energy monitor for measuring the energy of the electromagnetic radiation.

In another embodiment, a portion of the plurality of transducers is capable of transmitting ultrasound into the tissue, and a portion of the plurality of transducers is capable of receiving ultrasound emitted from the tissue, wherein the system is further capable of producing ultrasound images of the tissue.

The encasement is optionally positioned between the source and the support structure, with the encasement further including a window through which the electromagnetic radiation from the source passes to the support structure.

The system may also include a plurality of sources of electromagnetic radiation, wherein the electromagnetic radiation from each source is sufficient to induce a thermoacoustic response in the tissue positioned in the support structure, and wherein the plurality of sources are positioned to illuminate different portions of the tissue.

The support structure may or may not separate the tissue from acoustic coupling medium in the chamber. In certain embodiments, the system includes an acoustic coupling medium disposed in the chamber and having a speed of sound of 1450-1600 m/s.

Preferred transducers have a center frequency of 1 to 30 MHz and a bandwidth of greater than 50%. The encasement may include a spherical inner surface, e.g., wherein the plurality of acoustic transducers is positioned on the inner surface of the encasement so that the axis of maximum sensitivity of each transducer intersects the centroid of the sphere. Such a surface may have a radius of 80-150 mm. The encasement may also include a cylindrical section extending from the sphere equator to accommodate displacement of acoustic coupling medium by the introduction of the tissue to the support structure.

An exemplary source produces a pulse sequence of one or more pulses, each with an individual pulse length less than 500 nanoseconds, at a pulse rate greater than 1 Hertz. The energy per pulse is optionally greater than 0.03 mJ. The electromagnetic radiation is, for example, infrared, visible, UV, radio frequency, or microwave.

The system may also include a computer for generating an image or volumetric representation of the tissue from the thermoacoustic response. The support structure may include markings to show the field of view for thermoacoustic imaging. The portion of the support structure holding the tissue may or may not conform to the tissue. The portion of the support structure holding the tissue may also or alternatively be shaped to maintain the tissue in substantially the same orientation for thermoacoustic imaging.

The invention also features a method of producing a thermoacoustic image of a tissue by (a) providing a system for imaging as described herein; (b) placing the tissue in the support structure; (c) actuating the source to induce a thermoacoustic response in the tissue; (d) receiving ultrasound from the thermoacoustic response of the tissue at the plurality of acoustic transducers; and (e) generating a thermoacoustic image or volume from the received ultrasound.

Other features and advantages will be apparent from the following description, the drawings, and the claims.

### BRIEF DESCRIPTION OF THE DRAWINGS

FIG. 1 is an isometric view of an exemplary encasement.

FIG. 2 is a cut away section through an exemplary system, without external covers.

FIG. 3 shows an exemplary specimen-positioning tray.

FIG. 4 shows a system with an E-chain cable management system.

FIG. 5 shows the results of imaging a single absorbing point with the system employing laser illumination.

FIG. 6 shows a volume image derived from imaging an intact mouse in the system employing laser illumination.

### DETAILED DESCRIPTION OF THE INVENTION

A photo-acoustic system has been developed specifically for low volume imaging (including small animal imaging) with the specific aim of applications in the study of disease, the guidance of procedures, and the monitoring of therapies in the fields of academic research, pharmaceutical drug development, and clinical applications. Low volume imaging refers to imaging of a single organ or focal volume of interest and is differentiated from the more common ‘whole body’ imaging available with modalities such as: magnetic resonance (MR), x-ray computed tomography (CT), and positron emission tomography (PET) where large scan volumes covering multiple organs are available.

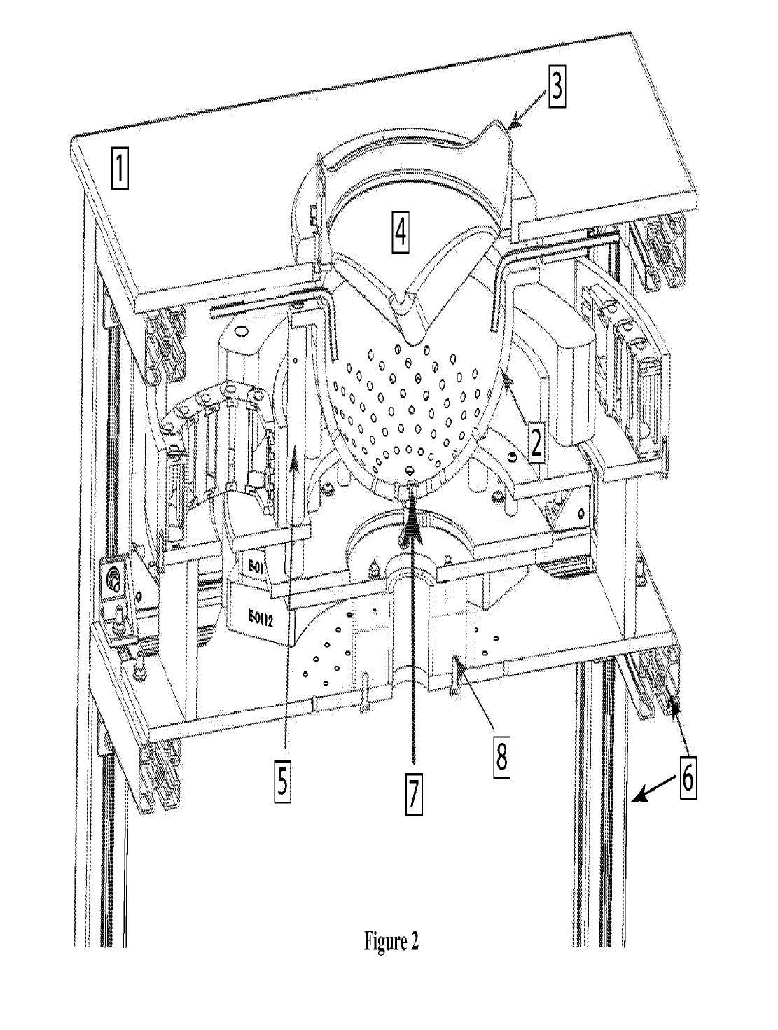

In its simplest embodiment, the system includes an electromagnetic radiation source, acoustic transducers, a support structure, and an encasement to which the transducers are attached. FIG. 2 shows a cut away section through a system without external covers. This system includes both moving and stationary parts. A table top [1] is attached to structural frame members [6] and provides a working surface that is stationary at all times. The encasement and plurality of acoustic transducers [2] are located beneath the table top and are attached to an electro-mechanical, computer controlled rotation stage [8] by way of support struts [5] to form a rotating assembly. The rotation stage has an unobstructed path through its axis of rotation [7] that allows an unimpeded path for illumination of the tissue from below the encasement. The tissue support structure [4] rests on the table top and remains stationary at all times during the data acquisition procedure. The support structure is attached to a handling apparatus [3] that allows for removal and positioning of the tray.

The system may further include various additional elements as described herein. The individual components of the system are discussed below. It will be understood that the system is constructed to provide for thermoacoustic imaging of a tissue located within it.

Electromagnetic Radiation Source

Any electromagnetic radiation source capable of producing a thermoacoustic response in a particular tissue may be employed. The radiation may be ionizing or non-ionizing, e.g., infrared, visible, ultraviolet, radio frequency (U.S. Pat. No. 6,633,774), or microwave (such as 10 MHz to 4 GHz). An exemplary source is a laser. The radiation may be pulsed, e.g., at greater than 1 Hz, or continuous. Pulse length may be less than 500 ns, and the energy per pulse may be less than 1 mJ, e.g., less than 0.03 mJ. The system may further include a monitor to measure the pulse energy.

In one embodiment, the one or more sources may be employed. When multiple sources are employed, they are typically positioned to illuminate a tissue at different locations. Sources may pass radiation through a window in the encasement. Alternatively, a source is positioned to illuminate from within or above the encasement. Combinations of sources directed from the bottom and top results in more uniform light distribution along the tissue being imaged. Multiple sources can be synchronized by using a common trigger signal and trigger delay, for all individual sources. In this manner, the cumulative energy of the individual sources will increase the thermoacoustic signal response from the tissue. Increased signal is generally desirable, particularly when increased sensitivity is required to detect trace materials or small changes in concentration of the absorber. Instead of using multiple sources, a single source can be used with the radiation split into multiple paths that will illuminate the tissue from multiple positions.

Encasement

The encasement is a structure to which acoustic transducers are attached and which houses an acoustic coupling medium. The medium is placed in the encasement to provide acoustic coupling between the transducers and a tissue located in a support structure, as described in more detail below.

The encasement may be of any shape suitable for transducers to receive ultrasound emitted from a tissue placed within it, e.g., spherical. For example, the transducers may be arranged in a spiral pattern within a portion of the inner surface of a hemispherical encasement, e.g., with a radius of 80-150 mm.

The encasement is typically filled with an acoustic coupling medium e.g., a liquid (such as water) or a gel. Acoustic coupling media are known in the art. The speed of sound (SOS) in the medium can be closely matched to SOS of the tissue being imaged. A medium having a speed of sound of 1450 to 1600 m/s is preferred. In one embodiment, water is combined with glycerol to produce a medium with the desired SOS. In some embodiments, the encasement includes a drainage hole in to allow for removal of liquids from the encasing and to facilitate cleaning and disinfection. The drainage hole is positioned so that it does not interfere with the detectors. The encasement typically also includes a volume (e.g., a cylindrical extension of a hemisphere) into which coupling media can be displaced when a support structure is inserted in the system, as discussed below.

The encasement may be constructed of conductive or non-conductive materials (which are preferred for use with radio and microwave frequencies). Engineered thermoplastics such as Delrin and Ultem are suitable encasing materials as they are chemically inert and machinable and have low water absorbance. As discussed above, the encasement may include a window (or otherwise be transparent) to radiation emitted from a source.

A temperature probe may also be installed on the encasement (or adjacent to it) to monitor and/or regulate the temperature of the acoustic coupling medium. Maintaining consistent temperature will result in consistent speed of sound through the coupling medium and may also reduce motion of the tissue being imaged. This is particularly relevant for imaging of animals. The temperature can be maintained through heaters located on or within the encasement. Alternatively, medium, e.g., water, can be exchanged between an external tank with constant temperature and the encasement. Preferably, medium is only exchanged between the external tank and the encasement between successive scans to prevent bubble formation during scans. Preferably, the temperature of the medium is matched to the normal physiological temperature of the tissue being imaged. In some embodiments, a temperature below physiological is advantageous. For example, in some small animal imaging applications (such as the mouse), a lower heart rate may be preferred and may be achieved by lowering the temperature of the liquid 1-5 degrees Celsius. Temperatures may also be lowered to maintain the integrity of an isolated tissue. The normal range of temperature for in vivo imaging for the medium will be 30-39 degrees Celsius.

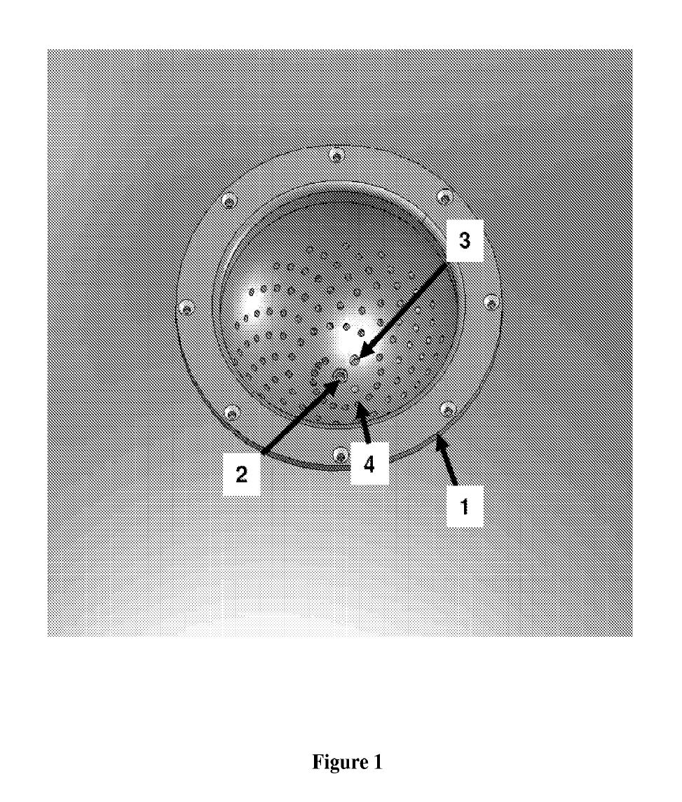

FIG. 1 is an isometric view of an exemplary encasement. The encasement [1] is machined, formed, or molded to provide the required geometry. The figure illustrates the pattern of machined holes into which the acoustic receivers are placed and form a spiral pattern as described in U.S. Pat. No. 5,713,356 and U.S. Pat. No. 6,102,857. A window [2], at the bottom of the encasement, provides an entry port through which electromagnetic radiation may be delivered to the tissue. A drainage hole [3] is also located in proximity of the lowest point of the encasing. A flexible hose, with a valve, is connected to the drainage hole by way of a fitting to allow the acoustic coupling media to be removed from the encasing. An additional hole in the encasing provides access for a temperature sensor to monitor the temperature of the acoustic coupling media.

Support Structure

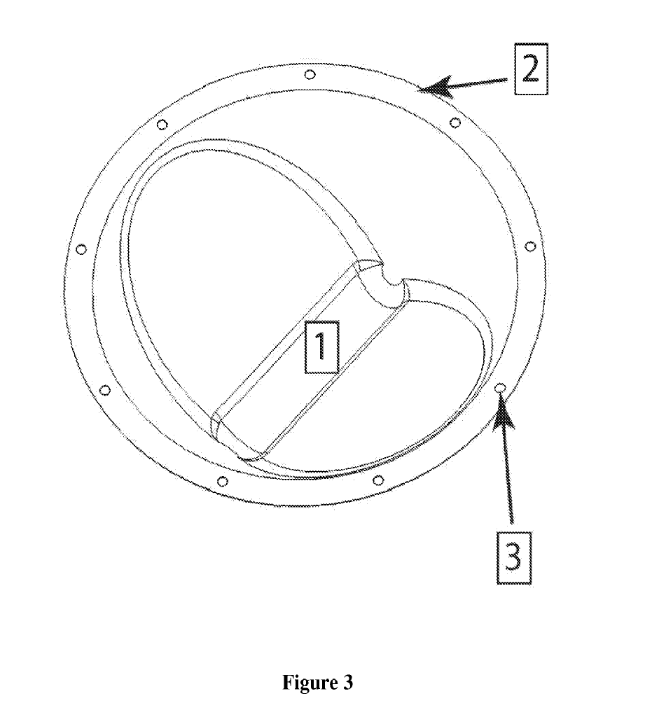

The support structure houses the tissue being imaged. The structure is placed in contact with acoustic coupling medium held in the encasement. Preferably, the support structure includes a portion that is able to conform to the shape of the tissue being imaged or is molded to hold the tissue in substantially the same orientation for thermoacoustic imaging, e.g., by approximating the shape of the tissue being imaged (FIG. 3). The support structure also positions the tissue appropriately in the system's field of view, i.e., the volume that can be thermoacoustically imaged. The height of the support structure may be adjustable, e.g., to allow the tissue to be centered vertically and/or horizontally in the system's field of view. Markings may be included on the support structure to assist in localizing the tissue in the field of view. The support structure may be removable from the rest of the system or may be hinged along one side of the system (or otherwise attached). Both of these approaches will facilitate cleaning of the encasement. The portion holding the tissue preferably prevents contact between the tissue and the acoustic coupling medium. The support structure may also include molded portions to accommodate non-imaged portions of a tissue, e.g., an arm, leg, animal tail, etc. The structure may further allow for the connection of catheters (e.g., arterial or venous) for delivery or removal of fluids to the tissue or other elements to the tissue, e.g., heart rate, breathing rate, or temperature.

The portion of the support structure that holds the tissue (which may be referred to herein as a cradle) may be removable and disposable. Alternately, this portion may be sterilized after each use. The portion holding the tissue can be rigid or deformable, preformed or flat. The acoustic impedance of the material employed in the portion housing the tissue is matched to the tissue. Additionally, the portion housing the tissue may have a high transmittance for the radiation being employed. The thickness of the portion holding the tissue is for example between 10 and 250 microns. Examples of suitable materials for the portion holding the tissue are: polycarbonate (e.g., Lexan®), polyethylene, perfluoroelastomer, polyethylene terephthalate, and plastic wrap (e.g., Saran®).

In another embodiment, the support structure allows a portion of the tissue in the path of illumination to be directly in contact with the coupling medium. In this embodiment, the support structure is not required to be transparent to the illuminating energy and the acoustic impedance of the support structure does not need to approximate the acoustic impedance of the tissue.

FIG. 3 shows an exemplary support structure. The cradle [1] is formed to approximate the geometry of the tissue of interest. The support structure has a horizontal rim [2] and screw holes [3] that allow it to be attached to the handling apparatus. Together with the handling apparatus, the support structure is inserted into the table top for the scanning procedure. The geometry of the support structure and cradle are of appropriate dimensions such that the tissue of interest is located at the effective field of view of each acoustic transducer in the encasement.

Acoustic Transducers

The system includes a plurality of acoustic transducers, e.g., at least 128, for receiving ultrasound produced thermoacoustically. The transducers may be arranged on the encasement as is known in the art, e.g., in a spiral pattern as disclosed in (U.S. Pat. No. 6,102,857). When the encasement has a spherical surface, the transducers may be arranged so that the axis of maximum sensitivity of each transducer intersects the centroid of the sphere. Exemplary transducers have center frequencies of 1 to 30 MHz and bandwidths of at least 50%.

One or more of the transducers may be used as an emitter of ultrasound, while one or more of the others are used as receivers for the production of an ultrasound image.

E-chains or other cable management systems may be used with the transducers to connect them to data storage and/or analysis components.

Additional Components

The system may also include a cover to enclose the tissue in conjunction with the encasement. Such a cover may also provide a structure for mounting electromagnetic radiation sources or optics to direct radiation to a tissue. The system may also include a protective shield to shield portions of a tissue from electromagnetic radiation.

Additionally, an optical camera, e.g., having sensitivity from 300 to 1064 nm, may be included and used to monitor the tissue during thermoacoustic imaging or to form an optical image based on: reflection, transmission, or emission, e.g., fluorescence, during the imaging procedure. The camera may be integrated into the cover above the tissue being scanned, lateral to the tissue, or external to the imaging system with the optical image of the tissue obtained using relay optics.

The system may further include a rotation stage to move the encasement relative to the tissue and/or radiation source. The stage rotates to provide thermoacoustic waveforms from multiple views. The rotation stage may have a hole through its vertical axis to provide an unimpeded light path to the window at the base of the encasing. The rotation stage may be manually driven or driven by a computer controlled drive system that allows for discrete increments, e.g., of 1 degree or less, or continuous rotation. The rotation stage may also include an encoder that allows for the recording of angular position at any given time.

The system may further include data storage and/or data analysis components. In one embodiment, the system includes a digital acquisition system that acquires and stores thermoacoustic response signals received by the transducers. The system may also include a computer that generates two-dimensional images or three-dimensional volumetric representations of the tissue based on the thermoacoustic responses received. The data storage and acquisition components and/or computer may also be used to storage and generate ultrasound images or volumes, when the transducers are used to transmit and received ultrasound.

The system may further include a table top that tilts (hinged on struts) so that the encasement surface may be accessed for cleaning and disinfecting; an optically opaque cover placed over the imaging area to provide shielding from stray laser light during imaging; or an interlock switch on the cover that connects to the laser to ensure no exposure to the imaging area when the cover is open.

Methods of Use

The system of the invention may be employed to produce thermoacoustic images and volumetric representations of a tissue, as is known in the art. Tissues imaged may be entire organisms, e.g., a plant, a mouse, rat, or rabbit; portions of an animal, e.g., a hand, foot, or breast; or material excised from an animal or grown in culture, e.g., a biopsy specimen or tissue implant.

### Examples

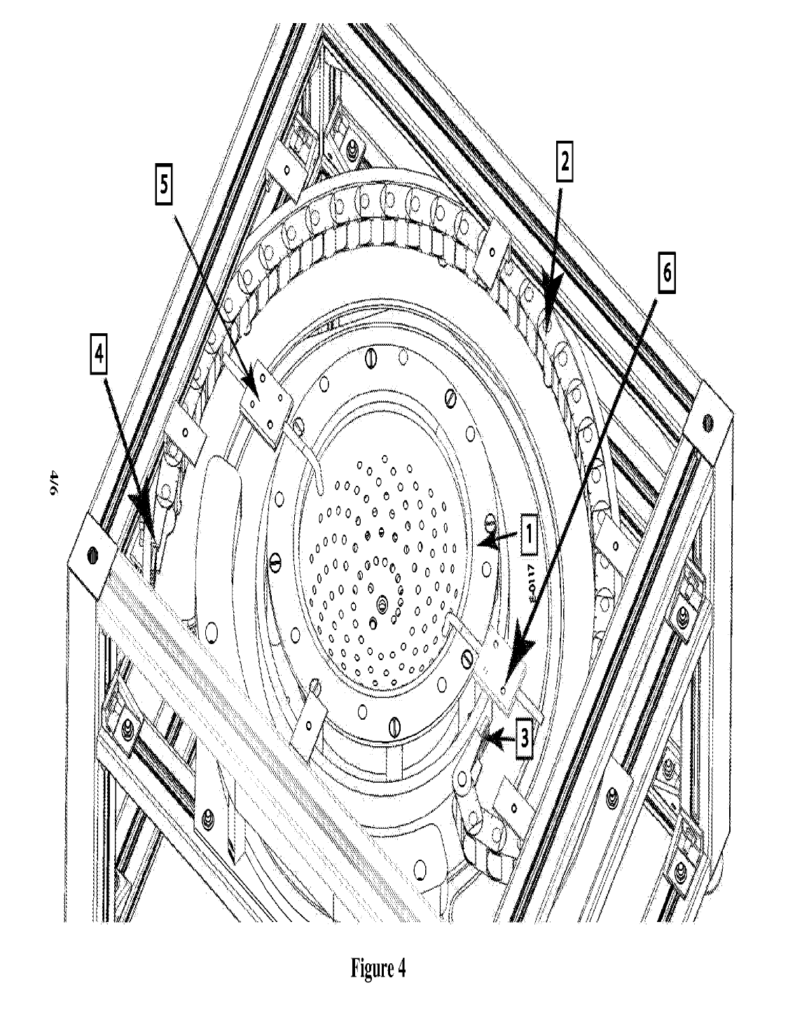

An exemplary system is described as follows. Any component specifically descried below may be employed with other components of the system and is generally applicable to the invention. FIG. 4 illustrates a system as viewed from above, without external covers. The acoustic transducers in the encasement [1] rotate through 360 degrees to provide multiple views of the thermoacoustic waveforms emitted from the tissue as it is illuminated. Each acoustic transducer has a pair of electrical wires (signal and ground). The pairs of electrical wires from all acoustic transducers in the encasement come together to form a cable. The cable is guided through the e-chain cable management system [2] between a fitting on the rotating portion of the scanner [3] and a fitting on the stationary scanner frame [4] allowing unencumbered motion of the cable within the photoacoustic scanner. An in-flow tube [5] delivers temperature controlled acoustic coupling media into the encasing, while an out-flow tube removes acoustic coupling media from the encasing and transfers it to an external temperature control unit. The combination of in-flow/out-flow tubes, an external pump, and temperature control unit allow for the acoustic coupling media to be at a constant and controlled temperature during the imaging procedure.

The energy source is a tunable OPO laser source capable of generating 40 mJ per pulse, at a wavelength of 300-1064 nm, with pulse duration <10 ns. The laser induces heating in the tissue being imaged. An optical chain including lenses, diffusers, filters, prisms, mirrors, and fiber optic cables is employed to relay the light emitted from the laser to the tissue. A beam splitter is used to provide two separate light sources for illuminating the tissue in the field of view. Alternatively, additional beam paths are incorporated with an integrating sphere with a photodiode to monitor the energy of each laser pulse. One beam path impinges on the animal, while the other (<5% of the total) is relayed to the integrating sphere (or alternate beam monitoring device) to quantify the light output per pulse. The energy of each pulse during a scan sequence, as measured by the beam monitor, is recorded as part of an acquisition sequence on the computer.

128 acoustic transducers are arranged within a hemispherical encasement (4″ radius) with an optical window at the base (entry port for light illumination from the bottom). The transducers (unfocused, flat front surface) are arranged in a spiral pattern. Each transducer has a pair of wires (signal and ground, groups of the ground leads come together into one lead). The signal and ground wires come together into a bundle with an outer sheath, making up a cable. The cable is approx. 2 meters in length and terminates in a 156 pin connector (standard ultrasound ITT/canon DL-1 connector). The DL-1 connector mates to a digital acquisition system (DAS) with 128 channels digitizing the input signals from each of the 128 transducers. The DAS has analog electronics with two amplifier stages providing gain 30 dB and digitizing at sample rates of 5, 10, 20, 40 MHz. An anti-aliasing filter employing a Hamming or Hamming window, with a user selectable cut-off frequency, is available in the gain—ND electronics to eliminate artifacts resulting from under-sampling. The signal is digitized and stored into a field-programmable gate array (FPGA) (24 bits/sample) with up to 2048 samples stored per transducer. Individual signals generated from multiple laser pulses may be averaged in the FPGA to provide increased signal to noise. Multiple DASs may be employed, e.g., with 256 or 512 detectors.

The number of pulses from the radiation source, the selection of the anti-aliasing filter, digitizing rate, and amplifier gain may be set from commands to the DAS from an acquisition computer through a universal serial bus (USB) connection.

The DAS has a trigger input. A pulse from the laser triggers the digitization. The waveform is amplified, digitized, averaged with the waveforms from other laser pulses, and stored in the FPGA. Once all of the laser pulses for a given position of the transducer geometry have been acquired and averaged, the resulting digitized waveform is transferred to the acquisition computer.

An ultrasound image is formed by using a single transducer element in the array as an emitter by placing an RF pulse on its signal line. The resulting signal returning from the tissue is recorded for all transducers in the encasement. The ultrasound transmit process may be repeated for all individual transducers in the array and for multiple rotational positions of the encasement. The recorded signals are used to form an ultrasound image of the tissue being imaged.

The encasement rests on a rotation stage. The stage rotates to provide thermoacoustic waveforms to be collected from multiple views. The rotation stage has a hole through its vertical axis to provide an unimpeded light path from the fiber optic to the glass entry window at the base of the encasement. The rotation stage is driven by a computer controlled drive system that allows for discrete increments or continuous rotation. The rotation stage has an encoder that allows for the recording of angular position at any given time.

The imaging area (FOV) is centered at the iso-center of the encasement. This iso-center can also be understood as the optimal point for imaging, given the placements of the transducers. The transducers are positioned in the encasement so that the central axes (perpendicular to the front faces of the transducers) intersect at the iso-center.

The encasement is hemi-spherical with vertical walls (cylindrical) rising (1.5″) from the equator rim. This provides capacity for coupling medium that will fill the encasement for acoustic coupling between the tissue imaged and each transducer.

The support structure holding the tissue is located above the encasement and has a hole (˜5″ radius). A deformable plastic, molded cradle (i.e., portion of the support structure that holds the tissue) is placed into the hole in the support structure. The deformable cradle is made of material with acoustical impedance close to or matching that of the coupling medium, e.g., water. The shape and geometry of the cradle allow the tissue to be located within the useful imaging FOV.

Light delivery is from the bottom of the encasement, through the window with a beam size so that the area of the laser pulse illuminating the animal is 1 square centimeter. Alternatively, light may be delivered from below and from above, wherein the light from above the specimen may illuminate the opposite surface (relative to the light from below). The above light is delivered by a fiber optic that may be manually positioned.

The height of the cradle may be adjusted vertically. A plane of laser light coming horizontally from the side can be used to determine the optimal height for the specimen. This optimal height can be identified by the laser light pointing at the iso-center (or other area of interest) of the tissue. Positioning the specimen in the horizontal plane is facilitated by markings on the support structure and cradle, which show the center of the FOV and the outer boundary of the FOV. The support structure and/or cradle portion has a shaped feature to accommodate the tail of a rodent being imaged to facilitate catheterization for injection or continuous infusion of contrast material. The encasement is filled with a liquid, e.g., water, to provide acoustic coupling from transducer to cradle. The tissue is coupled to the cradle with an acoustic coupling gel.

The system also includes a digital control unit having several functions: monitoring the energy of each laser pulse; control of a mechanical shutter, e.g., an electro-mechanical actuator to block the laser beam (the beam stop) and allow the laser to be conditioned without exposing the imaging area; rotation stage encoding to record the angular position of the stage; and temperature monitoring of the liquid filling the encasement.

The system also includes an acquisition computer to control data acquisition. A typical application sequence includes control over motion by sending commands to a motion control device to determine the angular position of the encasement; the laser by setting the pulse rate and wavelength through serial communication; the DAS control to determine the digitizing rate, filter function, gain, and number of pulses to average per transducer position through USB sets; and a micro-controller to control the beam stop monitor liquid temperature, and read the pulse energy.

The impulse response for each transducer is recorded. The characteristic functions are stored on the computer. Each signal that is digitized is deconvolved with the corresponding filter function for that transducer. The time derivative is computed (U.S. Pat. No. 5,713,356). The data for each transducer are back projected for each position of the transducer geometry (Kruger et al., Photoacoustic ultrasound (PAUS)—reconstruction tomography., Med. Phys. 22 (10), October, 1995, pp. 1605-1609). Image reconstruction is possible with 128 transducers and rotation of less than 180 degrees. Use of 256 transducers allows for use of 180 degrees of rotation for optimal sampling.

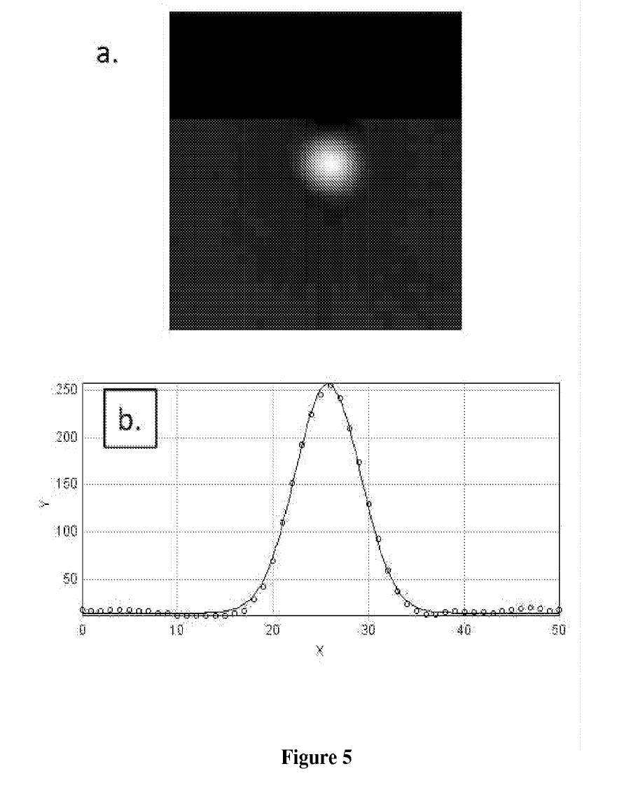

FIG. 5 illustrates the results of imaging a 200 micron absorbing test object. A 200 micron circle composed of highly absorbing printer ink was printed onto a thin, transparent, sheet of Mylar. The circle was placed at approximately the spherical center of the encasement and imaged with the photoacoustic system. The printer ink dot was illuminated by 7 ns pulses of light at a wavelength of 800 nm with 6 mJ of energy per pulse. The thermoacoustic waveforms emitted from the light absorbing circle were detected by 128 acoustic receivers in the encasing, digitized by a 128 channel digital acquisition system sampling the waveform at 20 Mega-Hertz, and stored on a computer. Thermoacoustic data were acquired for multiple views at 64 equally distributed rotational positions of the encasing over 360 degrees. The digitized data from all acoustic receivers, from all views, was reconstructed using the methodology as described in Kruger et al., Photoacoustic ultrasound (PAUS)—reconstruction tomography, Med. Phys. 22 (10), October, 1995, pp. 1605-1609. The resulting intensity image representing relative absorption is show in FIG. 5(a). An intensity profile of the reconstructed data through the center of the absorbing printer ink circle is shown in FIG. 5(b). The full width at half maximum for the profile plot is 280 microns.

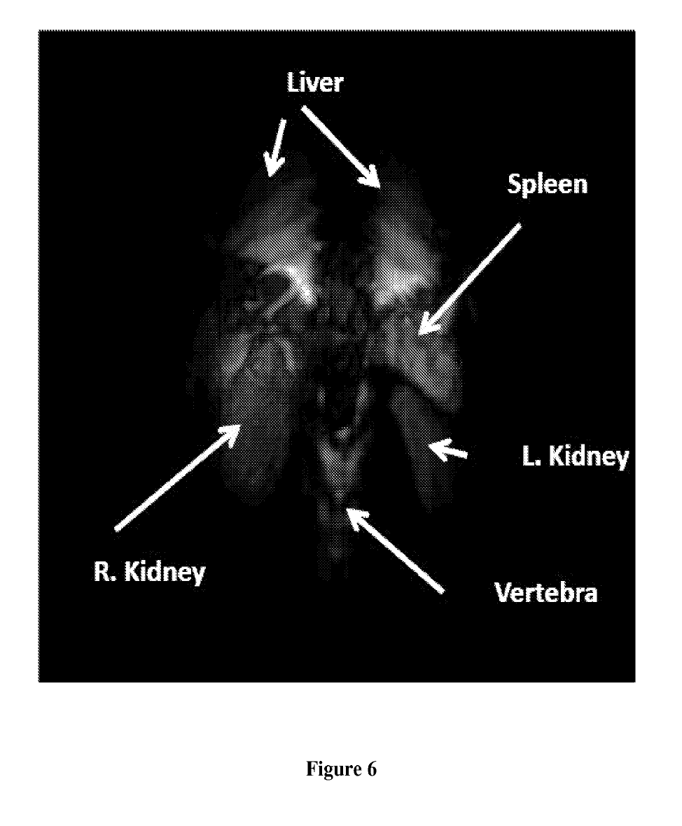

FIG. 6 illustrates a reconstructed photoacoustic volume derived from imaging an intact mouse with 7 ns laser pulses of light at 800 nm. Thermoacoustic waveforms were acquired at 64 equally spaced rotational positions of the encasing over a span of 360 degrees. The image represents a maximum intensity projection of a 3 mm coronal section through the abdomen of the mouse. A number of abdominal organs along with the lumbar vertebrae are clearly visible.

Other Embodiments

All publications, patents, and patent application publications mentioned herein are hereby incorporated by reference. Various modifications and variations of the described compounds of the invention will be apparent to those skilled in the art without departing from the scope and spirit of the invention. Although the invention has been described in connection with certain embodiments, it should be understood that the invention as claimed should not be unduly limited to such embodiments. Indeed, various modifications of the described modes for carrying out the invention that are obvious to those skilled in the relevant art are intended to be within the scope of the invention.

What is claimed is:

1. A system for imaging tissue comprising:
(i) a source of electromagnetic radiation;
(ii) an encasement comprising a plurality of acoustic transducers;
(iii) a support structure comprising a portion for holding a tissue; and
(iv) a chamber between the encasement and support structure for housing an acoustic coupling medium;
wherein electromagnetic radiation from the source is sufficient to induce a thermoacoustic response in the tissue positioned in the support structure, and the plurality of acoustic transducers are positioned to receive ultrasound from the thermoacoustic response of the tissue, and
wherein (a) the portion for holding the tissue has a thickness of less than 250 μm, and the acoustic impedance of the portion is matched to the tissue or (b) the portion allows for contact between the tissue and the acoustic coupling medium.

2. The system of claim 1, further comprising an optical camera positioned to monitor the tissue in the support structure.

3. The system of claim 2, wherein the camera is sensitive to light from 300-1064 nm.

4. The system of claim 1, further comprising an electro-mechanical motion control system for rotation of the encasement relative to the support structure.

5. The system of claim 4, wherein the motion control system is capable of rotating in discrete movements of 1 degree or less.

6. The system of claim 1, further comprising a digital acquisition system for acquiring and storing thermoacoustic response signals received by the plurality of transducers.

7. The system of claim 1, further comprising a temperature monitor and control system for maintaining a specified temperature of acoustic coupling medium in the chamber.

8. The system of claim 7, wherein the specified temperature is between 30 and 39° C.

9. The system of claim 1, further comprising a pulse energy monitor for measuring the energy of the electromagnetic radiation.

10. The system of claim 1, wherein a portion of the plurality of transducers is capable of transmitting ultrasound into the tissue, and a portion of the plurality of transducers is capable of receiving ultrasound emitted from the tissue, wherein the system is further capable of producing ultrasound images of the tissue.

11. The system of claim 1, wherein the encasement is positioned between the source and the support structure, and the encasement further comprises a window through which the electromagnetic radiation from the source passes to the support structure.

12. The system of claim 1, further comprising a plurality of sources of electromagnetic radiation, wherein the electromagnetic radiation from each source is sufficient to induce a thermoacoustic response in the tissue positioned in the support structure, and wherein the plurality of sources is positioned to illuminate different portions of the tissue.

13. The system of claim 1, wherein the support structure separates the tissue from acoustic coupling medium in the chamber.

14. The system of claim 1, further comprising an acoustic coupling medium disposed in the chamber and having a speed of sound 1450-1600 m/s.

15. The system of claim 1, wherein the plurality of acoustic transducer comprises at least 128.

16. The system of claim 1, wherein each of the plurality of acoustic transducers has a center frequency of 1 to 30 MHz and a bandwidth of greater than 50%.

17. The system of claim 1, wherein the encasement comprises a spherical inner surface.

18. The system of claim 17, wherein the plurality of acoustic transducers are positioned on the inner surface of the encasement so that the axis of maximum sensitivity of each transducer intersects the centroid of the sphere.

19. The system of claim 17, wherein the inner surface has a radius of 80-150 mm.

20. The system of claim 17, wherein the encasement is a hemisphere with a cylindrical section extending from the sphere equator to accommodate displacement of acoustic coupling medium by the introduction of the tissue to the support structure.

21. The system of claim 1, wherein the source produces a pulse sequence of one or more pulses, each with an individual pulse length less than 500 nanoseconds, at a pulse rate greater than 1 Hertz.

22. The system of claim 21, wherein the energy per pulse is greater than 0.03 mJ.

23. The system of claim 1, wherein the electromagnetic radiation is infrared, visible, UV, radio frequency, or microwave.

24. The system of claim 1, further comprising a computer for generating an image of the tissue from the thermoacoustic response.

25. The system of claim 1, further comprising a computer for generating a volumetric representation of the tissue from the thermoacoustic response.

26. The system of claim 1, wherein the support structure further comprises markings to show the field of view for thermoacoustic imaging.

27. The system of claim 1, wherein the portion of the support structure conforms to the tissue.

28. The system of claim 1, wherein the portion of the support structure is shaped to maintain the tissue in substantially the same orientation for thermoacoustic imaging.

29. A method of producing a thermoacoustic image of a tissue, said method comprising the steps of:
(a) providing a system for imaging tissue comprising:
(i) a source of electromagnetic radiation;
(ii) an encasement comprising a plurality of acoustic transducers;
(iii) a support structure comprising a portion for holding a tissue, wherein the portion has a thickness of less than 250 μm, and the acoustic impedance of the portion is matched to the tissue; and
(iv) a chamber between the encasement and support structure housing an acoustic coupling medium;
(b) placing the tissue in the support structure;
(c) actuating the source to induce a thermoacoustic response in the tissue;
(d) receiving ultrasound from the thermoacoustic response of the tissue at the plurality of acoustic transducers; and
(e) generating a thermoacoustic image or volume from the received ultrasound.
